(19) United States
(12) Patent Application Publication
(10) Pub. No.: US 2017/0130195 A1
(43) Pub. Date: May 11, 2017

(54) CELL CULTURE SUBSTRATE, MANUFACTURING METHOD THEREFOR, AND USE THEREOF

(71) Applicant: KOREA ADVANCED INSTITUTE OF SCIENCE AND TECHNOLOGY, Daejeon (KR)

(72) Inventors: Sung Gap IM, Daejeon (KR); Hak Rae LEE, Daejeon (KR); Sang Yong JON, Daejeon (KR); Min Suk CHOI, Daejeon (KR)

(21) Appl. No.: 15/318,134

(22) PCT Filed: Aug. 1, 2014
(86) PCT No.: PCT/KR2014/007113

§ 371 Date: Dec. 12, 2016

(30) Foreign Application Priority Data
Jun. 10, 2014 (KR) 10-2014-0069970

(51) Int. Cl. C12N 5/00; C08F 30/08; C09D 143/04; C12N 5/095

(52) U.S. Cl. CPC C12N 5/0068; C12N 5/0695; C08F 30/08; C09D 143/04; C12N 2533/30

(57) ABSTRACT

The present invention provides a cell culture substrate comprising a polymer formed of a cyclosiloxane compound and a manufacturing method therefor, and a method for preparing a cell spheroid type of cell aggregate or induced pluripotent stem cells using the cell culture substrate. The cell spheroid type of cell aggregate can be easily formed by culturing cells on the cell culture substrate of the present invention, and further, the cell culture substrate can be utilized as a cell culture platform for preparing the induced pluripotent stem cells.

### FIELD

The present invention relates to a cell culture substrate including a polymer formed of a cyclosiloxane compound, a manufacturing method therefor, and a method for manufacturing a cell spheroid type cell aggregate or induced pluripotent stem cells using the cell culture substrate.

### BACKGROUND

The culture of animal cells means a procedure in which biological tissues are excised from animal bodies, and then cells are isolated from the biological tissues and are allowed to proliferate in an incubator. There are a method of culturing cells attached to a substrate, such as an agar medium, a method of allowing cells to float on a culture liquid, and the like, according to the kind of cells, and various culture methods for obtaining three-dimensional culture cells have been more studied (Haycock J W., Methods Mol Biol, 695 (2011), pp. 1-15).

As an example of this culture, there is a method of using a scaffold, such as collagen, porous scaffold, or hydrogel (Chevallay B, Herbage D., Med Biol Eng Comput, 38(2) (2000), pp. 211-218; Ma L et al., Biomed Microdevices, 12(4) (2010), pp. 753-760; and Tibbitt M W et al., Biotechnol Bioeng, 103(4) (2009), pp. 655-663). As a method for being independent from a scaffold, there is a hanging-drop culture method for forming cell spheroids. The hanging-drop culture method has an advantage in that a scaffold is not used, but has a limit in that only micro-cellular tissues with 1 mm or less may be prepared.

Recently, a new culture method on the basis of magnetic levitation using magnetized hydrogels having RGD nucleotide sequences attached to cellular surfaces and nano/microparticle-sized material polymers (Souza G R et al., Nat Nanotechnol, 5(4) (2010), pp. 291-296). However, this culture method has a limit in that an artificial material should be attached to cell surfaces.

Meanwhile, induced pluripotent stem cells (iPCS) are stem cells which, unlike embryonic stem cells, do not use fertilized eggs or woman's eggs, and thus are free of ethical problems, and have a similar differentiation level to embryonic stem cells. The induced pluripotent stem cells are manufactured by applying genetic modification to somatic cells to induce stem cells having similar differentiation characteristics to embryonic stem cells, and, as methods therefor, there are a method of preparing embryonic stem cells by combining a nucleus isolated from a somatic cell and an egg with a nucleus removed therefrom, a method of introducing four genes into somatic cells, and a method of inducing differentiation ability through external environmental stimuli, which was recently announced.

Of existing methods, the nuclear substitution method has a low success rate, has a problem of not being free from bioethical controversies due to the use of eggs, and is indicated as a problem for immune responses. The research group, led by Shinya Yamanaka of Kyoto University in Japan who won the 2012 Nobel Prize in Physiology or Medicine, had succeeded in preparing induced pluripotent stem cells by introducing four gene expression factors in somatic cells, but such a manner does not resolve a disadvantage of increasing the occurrence of cancer as a side effect due to the use of viruses. In addition, since it takes at least six months to secure stability passing through a procedure of collecting cells and reprogramming the cells into stem cells, the method by Shinya Yamanaka is hard to effectively use in the treatment of cancer patients.

It has recently been announced in Japan that pluripotent cells differentiable into any cell can be induced by merely immersing somatic cells in a weak acid without genetic manipulation, but such a study is not a reliable result at the present time since there is controversy, such as falsifying experimental results.

Prior to this study, the research group in China has conducted a study about the preparation of induced pluripotent stem cells using seven small molecular compounds without genes, but this study has a limit in that the number of small molecular compounds introduced is greater compared with an existing method of using genes and the efficiency thereof is below even 1% like in existing methods of inducing induced pluripotent stem cells.

Throughout the entire specification, many papers and patent documents are referenced and their citations are represented. The disclosure of the cited papers and patent documents are entirely incorporated by reference into the present specification, and the level of the technical field within which the present invention falls and the details of the present invention are explained more clearly.

### DETAILED DESCRIPTION

### Technical Problem

The present inventors endeavored to solve the problems, and as a result, the present inventors formed cell spheroids known to be in an initial differentiation stage of induced pluripotent stem cells by culturing various cells, such as cancer cells, stem cells, and somatic cells, on a homopolymer or copolymer formed of a cyclosiloxane compound, and verified through the analysis of gene and protein expression that these cell spheroids were reprogrammed, and thus the polymer can be utilized as a platform for cell culture, and then the present inventors completed the present invention.

Accordingly, an aspect of the present invention is to provide a cell culture substrate.

Another aspect of the present invention is to provide a method for manufacturing a cell spheroid type cell aggregate.

Still another aspect of the present invention is to provide a method for manufacturing induced pluripotent stem cells.

Still another aspect of the present invention is to provide a method for manufacturing a cell culture substrate.

Other purposes and advantages of the present invention will become more obvious with the following detailed description of the invention, claims, and drawings.

### Technical Solution

In accordance with an aspect of the present invention, there is provided a cell culture substrate including a polymer formed of a cyclosiloxane compound.

The present inventors endeavored to solve the problems, and as a result, the present inventors formed cell spheroids known to be in an initial differentiation stage of induced pluripotent stem cells by culturing various cells, such as cancer cells, stem cells, and somatic cells, on a homopolymer or copolymer formed of a cyclosiloxane compound, and verified through the analysis of gene and protein expression that these cell spheroids were reprogrammed, and thus the polymer can be utilized as a platform for cell culture.

As used herein, the expression “cell culture substrate including a polymer formed of a cyclosiloxane” is used to mean not only that the polymer formed of a cyclosiloxane corresponds to a portion of a cell culture substrate (e.g., a cell culture substrate surface-coated with the polymer), but also that a solid polymer per se formed of a cyclosiloxane may be used as a cell culture substrate.

According to the present invention, the shape of the cell culture substrate is not limited since the cell culture substrate is sufficient as long as it provides any space capable of culturing cells therein. For example, the cell culture substrate may have a shape of a dish (culture dish), a petri dish or plate (e.g., 6-well, 24-well, 48-well, 96-well, 384-well, or 9600-well microtiter plate, microplate, dip well plate, or the like), a flask, a chamber slide, a tube, a cell factory, a roller bottle, a spinner flask, a hollow fiber, a micro carrier, a bead, or the like.

In addition, any material having supportability may be used as the cell culture substrate without limitation, and for example, plastic (e.g., polystyrene, polyethylene, polypropylene, or the like), metal, silicon, and glass may be used as a cell culture substrate. The structure of the cell culture substrate according to an embodiment of the present invention is shown in FIG. 1.

As used herein, the expression “polymer formed of a cyclosiloxane” is used as a meaning including all of: (i) a homopolymer formed by polymerizing cyclohexane compounds of the same kind; (ii) a copolymer formed by polymerizing cyclosiloxane compounds of different kinds; and (iii) a copolymer formed by polymerizing cyclohexane compounds of the same kind or different kinds and another monomer compound. As used herein, the copolymer may be a random copolymer, a block copolymer, an alternating copolymer, or a graft copolymer, but is not limited thereto.

Therefore, according to an embodiment of the present invention, the polymer formed of a cyclosiloxane compound is a homopolymer formed of cyclosiloxane compounds of the same kind.

According to another embodiment, the polymer formed of the cyclosiloxane compound is a copolymer formed of a first monomer, which is the cyclosiloxane compound, and a second monomer polymerizable with the first monomer. As verified in the following examples, a cell spheroid type cell aggregate can be manufactured by culturing cells on the copolymer including a cyclosiloxane compound (see FIG. 10).

According to a particular embodiment, the second monomer is a cyclosiloxane compound different from the first monomer (a copolymer is formed of cyclosiloxane compounds of different kinds).

According to another embodiment, the second monomer is a compound having a carbon double bond for the polymerization with the first monomer. Here, the first monomer may also have a carbon double bond for the polymerization with the second monomer. For example, the second monomer compound may be selected from the group consisting of siloxanes having a vinyl group (e.g., hexavinyldisiloxane, tetramethyldisiloxane, etc.), methacrylate-based monomers, acrylate-based monomers, aromatic vinyl-based monomers (e.g., divinyl benzene, vinyl benzoate, styrene, etc.), acrylamide-based monomers (e.g., N-isopropyl acrylamide, N,N-dimethyl acrylamide, etc.), maleic anhydrides, silazanes or cyclosilazanes having a vinyl group (e.g., 2,4,6-trimethyl-2,4,6-trivinylcyclosilazane, etc.), C₃₋₁₀ cycloalkanes having a vinyl group (e.g., 1,2,4-trivinylcyclohexane, etc.), vinylpyrrolidones, 2-(methacryloyloxy)ethyl acetoacetate, 1-(3-aminopropyl)imidazole, vinylimidazoles, vinylpyridines, silanes having a vinyl group (e.g., allyltrichlorosilane, acryloxy methyltrimethoxysilane, etc.) and combinations thereof.

Examples of the methacrylate-based monomer include methacrylate, methacrylic acid, glycidyl methacrylate, perfluoro methacrylate, benzyl methacrylate, 2-(dimethylamino)ethyl methacrylate, furfuryl methacrylate, 3,3,4, 4,5,5,6,6,7,7,8,8,9,9,10,10,10-heptadecafluorodecyl methacrylate, hexyl methacrylate, methacrylic anhydride, pentafluorophenyl methacrylate, propargyl methacrylate, tetrahydrofurfuryl methacrylate, butyl methacrylate, methacryloyl chloride, and di(ethylene glycol)methyl ester methacrylate.

Examples of the acrylate-based monomer include acrylate, 2-(dimethylamino)ethyl acrylate, ethylene glycol diacrylate, isobornyl acrylate, 1H,1 H,2H,2H,-perfluorodecyl acrylate, tetrahydrofurfuryl acrylate, poly(ethylene glycol) diacrylate, 1H,1H,7H-dodecafluoroheptyl acrylate and propargyl acrylate.

The copolymer of the present invention may further contain, in addition to the monomers described herein, and other monomers, as comonomers.

According to an embodiment of the present invention, the cyclosiloxane compound may be contained at least 50% in the copolymer. According to a specific embodiment, the cyclosiloxane compound is contained at 60% or more, 70% or more, 80% or more, or 90% or more in the copolymer. Such a content is determined on the basis of a flow rate (unit: sccm), and 90% means the content of the cyclosiloxane compound contained in the copolymer formed by making respective monomers to flow at a flow rate of 9: 1 (cyclosiloxane compound: another monomer), 80% means at a flow rate of 8:1, 70% means at a flow rate of 7:1, and 60% means at a flow rate of 6:1.

As used herein, the term “cyclosiloxane compound” is used to cover compounds which have a cyclosiloxane structure as a base frame and have a functional group (e.g., alkyl group, alkenyl group, etc.) at the silicon atom position.

According to an embodiment of the present invention, the cyclosiloxane compound is represented by chemical formula 1 below.

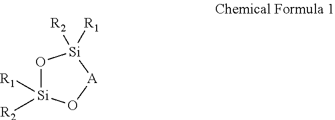

wherein

A is

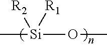

(n=an integer of 1-8);

R₁'s are each independently hydrogen or C₂₋₁₀ alkenyl (provide that at least two of R₁'s are C₂₋₁₀ alkenyl); and

R₂'s are each independently hydrogen, C₁₋₁₀ alkyl, C₂₋₁₀ alkenyl, halo, a metal element, C₅₋₁₄ heterocycle, C₃₋₁₀ cycloalkyl, or C₃₋₁₀ cycloalkenyl.

As used herein, the term “alkyl” refers to a straight or branched chain, substituted or unsubstituted saturated hydrocarbon group, and for example, includes methyl, ethyl, propyl, isobutyl, pentyl, hexyl, and the like. C₁-C₁₀ alkyl means an alkyl group having an alkyl unit with 1 to 10 carbon atoms, which does not include the number of carbon atoms on the substituent when C₁-C₁₀ alkyl is substituted.

According to an embodiment of the present invention, C₁-C₁₀ alkyl herein may be C₁-C₈ alkyl, C₁-C₇ alkyl, or C₁-C₆ alkyl.

As used herein, the term “alkenyl” refers to a straight or branched chain, substituted or unsubstituted unsaturated hydrocarbon group having a predetermined number of carbon atoms, and for example, includes vinyl, propenyl, allyl, isopropenyl, butenyl, isobutenyl, t-butenyl, n-pentenyl, and n-hexenyl. C₂₋₁₀ alkenyl means an alkenyl group having an alkenyl unit with 2 to 10 carbon atoms, which does not include the number of carbon atoms on the substituent when C₂-C₁₀ alkenyl is substituted.

According to an embodiment of the present invention, C₂₋₁₀ alkenyl herein may be C₂₋₈ alkenyl, C₂₋₆ alkenyl, C₂₋₅ alkenyl, C₂₋₄ alkenyl, or C₂₋₃ alkenyl.

According to an embodiment of the present invention, at least three of R₁'s are C₂₋₁₀ alkenyl.

According to an embodiment of the present invention, the cyclosiloxane has (n+1) or (n+2) C₂₋₁₀ alkenyl at the R₁ positions. For example, when n is 2, the compound of chemical formula 1 is cyclotetrasiloxane having three or four C₂₋₁₀ alkenyl groups at the R₁ positions. These alkenyl groups are involved in a polymerization reaction.

As used herein, the term “halo” refers to a halogen element, for example, includes fluorine, chlorine, bromine, and iodine.

As used herein, the term “metal element” refers to an element for metallic simple substances, such as alkali metal elements (Li, Na, K, Rb, Cs, Fr), alkaline earth metal elements (Ca, Sr, Ba, Ra), aluminum group elements (Al, Ga , In, Tl), tin group elements (Sn, Pb), currency metal elements (Cu, Ag, Au), zinc group elements (Zn, Cd, Hg), rare earth elements (Sc, Y, 57-71), titanium group elements (Ti, Zr, Hf), vanadium group elements (V, Nb, Ta), chromium group elements (Cr, Mo, W), manganese group elements (Mn, Tc, Re), iron group elements (Fe, Co , Ni), platinum group elements (Ru, Rh, Pd, Os, Ir, Pt), and actinide elements (89-103).

As used herein, the term “heterocycle” means a monocyclic or bicyclic 5-membered to 14-membered heterocyclic ring which is saturated partially or completely. N, O, and S are examples of a heteroatom. Examples of C₅₋₁₄ heterocycle include pyrrole, furan, thiophene, imidazole, pyrazole, oxazole, isoxazole, thiazole, isothiazole, tetrazole, 1,2,3,5-oxathiadiazole-2-oxide, triazolone, oxadiazolone, isoxazolone, oxadiazolidinedione, 3-hydroxypyrro-2,4-dione, 5-oxo-1,2,4-thiadiazole, pyridine, pyrazine, pyrimidine, indole, isoindole, indazole, phthalazine, quinoline, isoquinoline, quinoxaline, quinazoline, cinnoline, and carboline.

As used herein, the term “cycloalkyl” means a cyclic hydrocarbon radical, and includes cyclopropyl, cyclobutyl, and cyclopentyl. C₃₋₁₀ cycloalkyl means cycloalkyl having 3 to 10 carbon atoms and forming a ring structure, which does not include the number of carbon atoms on the substituent when C₃-C₁₀ cycloalkyl is substituted.

According to an embodiment of the present invention, C₁-C₁₀ cycloalkyl herein may be C₁-C₈ cycloalkyl, C₁-C₇ cycloalkyl, or C₁-C₆ cycloalkyl.

As used herein, the term “cycloalkenyl” means a cyclic hydrocarbon group having at least one double bond, and examples thereof include cyclopentene, cyclohexene, and cyclohexadiene. C₃₋₁₀ cycloalkenyl means cycloalkenyl having 3 to 10 carbon atoms and forming a ring structure, which does not include the number of carbon atoms on the substituent when C₃-C₁₀ cycloalkenyl is substituted.

According to an embodiment of the present invention, C₂₋₁₀ cycloalkenyl herein may be C₂₋₈ cycloalkenyl, C₂₋₆ cycloalkenyl, C₂₋₆ cycloalkenyl, C₂₋₄ cycloalkenyl, or C₂₋₃ cycloalkenyl.

According to an embodiment of the present invention, R₂'s are each independently hydrogen, C₁₋₁₀ alkyl, or C₂₋₁₀ alkenyl. According to a specific embodiment, at least two or at least three of R₂'s may be C₁₋₁₀ alkyl or C₂₋₁₀ alkenyl. According to a specific embodiment, the cyclosiloxane may be (n+1) or (n+2) C₁₋₁₀ alkyl or C₂₋₁₀ alkenyl at the R₂ positions.

According to an embodiment of the present invention, n is an integer of 1-7, an integer of 1-6, an integer of 1-5, an integer of 1-4, or an integer of 1-3.

According to an embodiment of the present invention, the cyclosiloxane compound is selected from the group consisting of

2,4,6,8-tetra(C₁₋₁₀)alkyl-2,4,6,8-tetra(C₂₋₁₀)alkenylcyclotetrasiloxane,
1,3,5-tri(C₁₋₁₀)alkyl-1,3,5-tri(C₂₋₁₀)alkenylcyclotrisiloxane,
1,3,5,7-tetra(C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
1,3,5-tri(C₁₋₁₀)alkyl-1,3,5-tri(C₂₋₁₀)alkenylcyclotrisiloxane,
1,3,5,7-tetra(C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
1,3,5-tri(C₁₋₁₀)alkyl-1,3,5-tri(C₂₋₁₀)alkenylcyclotrisiloxane,
1,3,5,7-tetra(C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
hexa(C₂₋₁₀)alkenylcyclotrisiloxane,
octa(C₂₋₁₀)alkenylcyclotetrasiloxane, deca(C₂₋₁₀)alkenylcyclopentasiloxane, and a combination thereof.

According to a specific embodiment, the cyclosiloxane compound is selected from the group consisiting of

2,4,6,8-tetra(C₁₋₆)alkyl-2,4,6,8-tetra(C₂₋₄)alkenylcyclotetrasiloxane (e.g., 2,4,6,8-tetramethyl-2,4,6,8-tetravinylcyclotetrasiloxane),
1,3,5-tri(C₁₋₆)alkyl-1,3,5-tri(C₂₋₄)alkenylcyclotrisiloxane (e.g., 1,3,5-triisopropyl-1,3,5-trivinylcyclotrisiloxane),
1,3,5,7-tetra(C₁₋₆)alkyl-1,3,5,7-tetra(C₂₋₄)alkenylcyclotetrasiloxane (e.g., 1,3,5,7-tetraisopropyl-1,3,5,7-tetravinylcyclotetrasiloxane),
1,3,5,7,9-penta(C₁₋₆)alkyl-1,3,5,7,9-penta(C₂₋₄alkenylcyclopentasiloxane (e.g., 1,3, 5,7,9-pentaisopropyl-1,3,5,7,9-pentavinylcyclopentasiloxane),
1,3,5-tri(C₁₋₆)alkyl-1,3,5-tri(C₂₋₄)alkenylcyclotrisiloxane (e.g., 1,3,5-tri-sec-butyl-1,3,5-trivinylcyclotrisiloxane),
1,3,5,7-tetra(C₁₋₆)alkyl-1,3,5,7-tetra(C₂₋₄)alkenylcyclotetrasiloxane (e.g., 1,3,5,7-tetra-sec-butyl-1,3,5,7-tetravinylcyclotetrasiloxane),
1,3,5,7,9-penta(C₁₋₆)alkyl-1,3,5,7,9-penta(C₂₋₄)alkenylcyclopentasiloxane (e.g., 1,3, 5,7,9-penta-sec-butyl-1,3,5,7,9-pentavinylcyclopentasiloxane),
1,3,5-tri(C₁₋₆)alkyl-1,3,5-tri(C₂₋₄)alkenylcyclotrisiloxane (e.g., 1,3,5-tri ethyl-1,3,5-trivinylcyclotrisiloxane),
1,3,5,7-tetra(C₁₋₆)alkyl-1,3,5,7-tetra(C₂₋₄)alkenylcyclotetrasiloxane (e.g., 1,3,5,7-tetraethyl-1,3,5,7-tetravinylcyclotetrasiloxane),
1,3,5,7,9-penta(C₁₋₆)alkyl-1,3,5,7,9-penta(C₂₋₄)alkenylcyclopentasiloxane (e.g.,
1,3,5,7,9-pentaethyl-1,3,5,7,9-pentavinylcyclopentasiloxane),
hexa(C₂₋₄)alkenylcyclotrisiloxane (e.g., hexavinylcyclotrisiloxane),
octa(C₂₋₄)alkenylcyclotetra siloxane (e.g., octavinylcyclotetrasiloxane),
deca(C₂₋₄)alkenylcyclo pentasiloxane (e.g., decavinylcyclopentasiloxane), and a combination thereof.

According to an embodiment of the present invention, the cell culture substrate is a cell culture substrate for manufacturing a cell spheroid type cell aggregate. The differentiated or undifferentiated cells may be cultured on the cell culture substrate of the present invention, thereby forming a spheroid type cell aggregate.

According to an embodiment of the present invention, the cell culture substrate is a cell culture substrate for manufacturing induced pluripotent stem cells. The differentiated cells, such as somatic cells, germ cells, and cancer cells, may be cultured on the cell culture substrate of the present invention, and the induced pluripotent stem cells may be induced (formed) from the differentiated cells.

According to an embodiment of the present invention, the polymer formed of the cyclosiloxane compound may be coated on the cell culture substrate. The surface coating of the polymer may be attained by a chemical vapor deposition method that is well known in the art.

According to an embodiment of the present invention, the cell culture substrate may be provided in a culture device (culture apparatus) for cell culture.

According to an embodiment of the present invention, the cell culture substrate has a water contact angle of 90 degrees (e.g., 90-100 degrees).

In accordance with another aspect of the present invention, there is provided a method for manufacturing a cell spheroid type cell aggregate, the method including a step for culturing cells on the cell culture substrate of the present invention.

In accordance with still another aspect of the present invention, there is provided a method for manufacturing induced pluripotent stem cells, the method including a step for culturing differentiated cells on the cell culture substrate of the present invention.

According to an embodiment of the present invention, the culture of cells is carried out in culture media. That is, the culture of cells is carried out on the polymer formed of a cyclosiloxane in the presence of culture media. As used herein, the term “culture media” means a culture liquid capable of supporting the survival of cells in in vitro culture conditions, and includes all the ordinary culture media that are suitable for cell culture and used in the art. In addition, the culture media and culture conditions may be differently selected depending on the kind of cells. As a culture medium used for the culture, for example, a cell culture minimum medium (CCMM) may be used, and the cell culture minimum medium generally contains a carbon source, a nitrogen source, and trace element components. Examples of the cell culture minimum medium include DMEM (Dulbecco's Modified Eagle's Medium), MEM (Minimal essential Medium), BME (Basal Medium Eagle), RPMI1640, F-10, F-12, aMEM (a Minimal essential Medium), GMEM (Glasgow's Minimal essential Medium), and Iscove's Modified Dulbecco's Medium.

The cell culture method or culture conditions may employ an ordinary cell culture method or culture conditions per se excluding that the cell culture substrate of the present invention is used. The culture method and culture conditions of animal cells are shown in Animal Cell Culture : A Practical Approach, Third edition (Masters, Oxford University Press, 2000), and reagents and kits for cell culture are accessible from Sigma-Aldrich, Invitrogen/GIBCO, Clontech, and Stratagene.

The cell culture may be carried out until the cell spheroid type cell aggregate is formed, and for example, the cell culture may be carried out for, for example, 10-60 hours (e.g., 10-48 hours or 20-48 hours).

According to an embodiment of the present invention, the differentiated cells are somatic cells or cancer cells. The somatic cells encompass all the cells, excluding germ cells, out of cells constituting the living body. According to a specific embodiment, the somatic cells are somatic cells from mammals (e.g., mouse, rat, rabbit, pig, sheep, goat, cow, monkey, and human being).

According to an embodiment of the present invention, for the manufacture of the cell spheroid type cell aggregate, the cells cultured on the cell culture substrate of the present invention are differentiated cells (e.g., somatic cells, germ cells, cancer cells) or undifferentiated cells (e.g., stem cells, such as mesenchymal cells and adipose-derived stem cells).

According to an embodiment of the present invention, the cell spheroids express SOX2, OCT4, or Nanog.

According to an embodiment of the present invention, the cell spheroids express β-cathenin and survivin protein at a higher level compared with adhering cells.

The following examples verified that, after a cell spheroid type cell aggregate was formed by culturing somatic cells on a cell culture substrate in which a polymer thin film has been deposited, the expression aspects of genes and proteins of the cell spheroid were different from those in original cells, and thus it was verified that cells were reprogrammed (see FIGS. 3 to 8). In addition, as a result of culturing mouse-derived fibroblasts on the cell culture substrate, it was verified that, OCT4 and Nanog, which are genes that are necessarily expressed in undifferentiated pluripotent stem cells, were expressed therein (see FIGS. 5 and 8), and the expression of SOX2, which is a gene that is associated with multipotency in the gene level, was verified through RT-PCR (see FIG. 6).

As examples of the method for confirming the formation of induced pluripotent stem cells (method of confirming cell multipotency), AP staining, cell fluorescence staining, stem cell-specific gene expression and quantitative PCR analysis of viral gene silencing, chimeric formation analysis, and germline transmission detection may be used.

In accordance with still another aspect of the present invention, there is provided a method for manufacturing the cell culture substrate of the present invention, the method including a step for forming a polymer thin film containing a cyclosiloxane compound as a monomer on a substrate through deposition.

According to an embodiment of the present invention, the polymer thin film is formed on the substrate through chemical vapor deposition. For example, the polymer thin film may be formed on the substrate through initiated chemical vapor deposition (iCVD) using an initiator. Since iCVD does not use an additive or a solvent in the entire process, the formed polymer thin film is very high, and thus a harmful solvent is not used when iCVD is applied to cell culture. The deposition by iCVD may be carried out for 30-60 minutes while the surface temperature of the substrate in which the polymer thin film is to be deposited is 35-45° C. and the pressure in the chamber in a reactor is 200-250 mbar.

### Advantageous Effects

Features and advantages of the present invention are summarized as follows:

(i) The present invention provides: a cell culture substrate including a polymer formed of a cyclosiloxane compound, a manufacturing method therefor, and a method for manufacturing a cell spheroid type cell aggregate or induced pluripotent stem cells using the cell culture substrate.

(ii) The cell spheroid type cell aggregate can be easily formed by culturing cells on the cell culture substrate of the invention, and further, the cell culture substrate can be utilized as a cell culture platform for manufacturing induced pluripotent stem cells.

### BRIEF DESCRIPTION OF THE DRAWINGS

FIG. 1 is a schematic view of a cell culture substrate according to an embodiment of the present invention.

FIG. 2 shows results of measuring surface contact angles with respect to a cell culture substrate before and after deposition.

FIG. 3 shows images of cell spheroids formed by culturing cells on the cell culture substrate of the present invention.

FIGS. 4 and 5 illustrate analysis results of cell spheroids through immunostaining. Scale bars indicate 100 μm.

FIG. 6 illustrates gene analysis results of cell spheroids.

FIG. 7 illustrates protein analysis results of cell spheroids.

FIG. 8 verifies that Nanog was expressed in cell spheroids. Scale bars indicate 100 μm.

FIG. 9 shows images of cell spheroids formed by culturing cells on each of cell culture substrates surface-coated with polymers formed of various cyclosiloxane compounds.

FIG. 10 shows images of cell spheroids formed by culturing cells on each of the cell culture substrates surface-coated with various copolymers formed of cyclosiloxane compounds.

### MODE FOR CARRYING OUT THE INVENTION

Hereinafter, the present invention will be described in detail with reference to examples. These examples are only for illustrating the present invention more specifically, and it will be apparent to those skilled in the art that the scope of the present invention is not limited by these examples.

### EXAMPLES

### Example 1

### Manufacturing of Cell Culture Substrate Surface-Coated with Polymer Thin Film

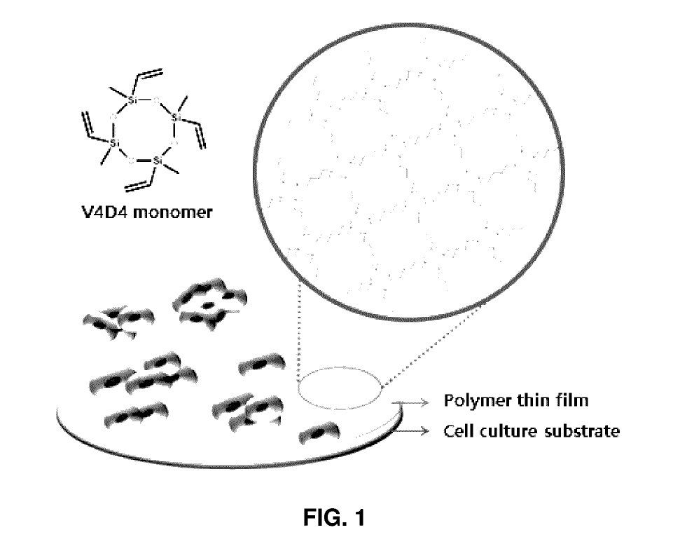

As shown in FIG. 1, into a monomer container of a initiated chemical vapor deposition reactor (iCVD, Daeki Hi-Tech Co., Ltd), 2,4,6,8-tetramethyl-2,4,6,8-tetravinylcyclotetrasiloxane (V4DA, Sigma-Aldrich) as a monomer was put, and then heated at 70° C. The tert-butyl peroxide (TBPO, Sigma-Aldrich) as an initiator was put into an initiator container, and then maintained at room temperature. The deposition was carried out using a polystyrene cell culture plate as a substrate. At the V4D4 deposition, the deposition was carried out for 40 minutes while the V4D4 monomer and TBPO were allowed to flow into the chemical vapor deposition reactor at a rate of 1:1 (flow rate base, unit: sccm), the filament temperature in the reactor is 200° C., the substrate temperature in the reactor is 40° C., and the chamber pressure in the reactor is 200 mbar, so that a cell culture substrate having pV4D4 (poly-V4D4) deposited with about 200 nm thickness was manufactured.

### Example 2

### Measurement of Contact Angle

After the polymer thin film was deposited, 8 μl of distilled water droplet for each measurement was dropped on a surface of the substrate, and the surface contact angle with respect to the substrate was measured using a contact angle measurement apparatus (Phoenix series, S. E. O. Co. Ltd).

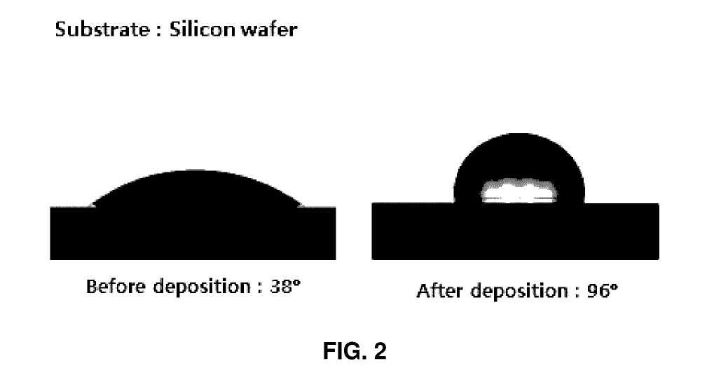

As a result, as shown in FIG. 2, when comparing images of contact angles before and after the deposition of the polymer thin film on the silicon wafer surface, it was verified that the contact angle was varied after the deposition as the polymer thin film was uniformly coated on the surface of the substrate (FIG. 2).

### Example 3

### Formation of Cell Spheroids

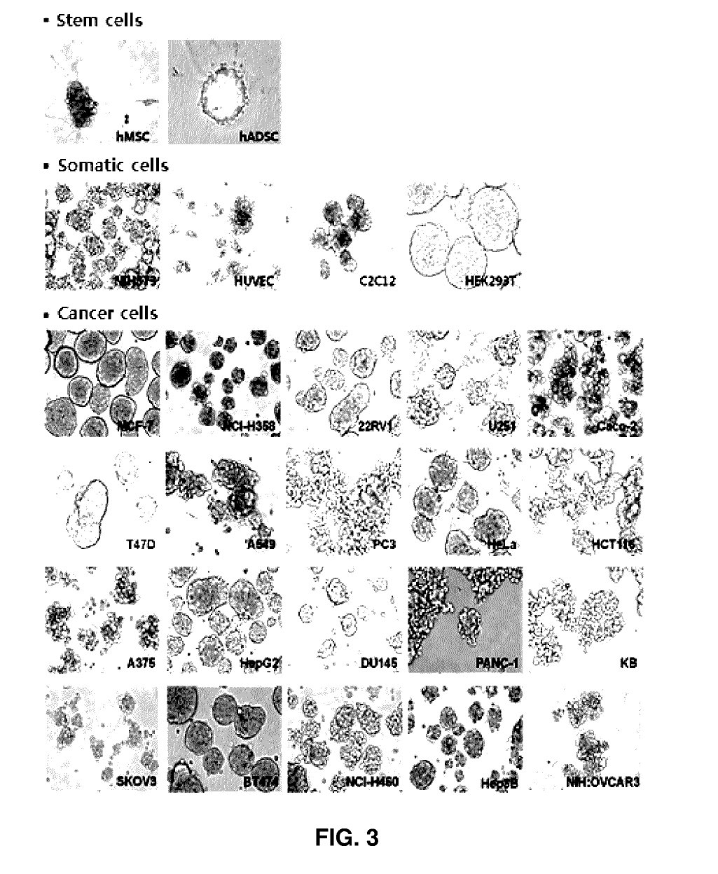

Cells were cultured on the cell culture substrate (dish) including the deposited polymer thin film manufactured in example 1, and then it was checked whether or not cell spheroids were formed. The used cells were cancer cells, stem cells, and somatic cells derived from humans, and somatic cells and cancer cells derived from mice, as shown in FIG. 3. The cancer cells were cultured using a medium in which 10% (v/v) fetal bovine serum (Thermo) and 5% of 1% (w/v) penicillin/streptomycin (Gibco) were supplemented to the cell culture minimum medium (CCMM). The somatic cells, such as NIH3T3, and stem cells, such as human mesenchymal stem cells (hMSC) and human adipose-derived stem cells (hADSC), were cultured using the media (Gibco) having compositions shown in table 1 (for human stem cells) and table 2 (for mouse stem cells) below. The culture was conducted for 24 hours.

TABLE 1

| Reagent | Final concentration | | Volume in 100 mL | |
|---|---|---|---|---|
| KnockOut ™ DMEM | 1X | | 78 | mL |
| KnockOut ™ SR | 20% | | 20 | mL |
| NEAA* | 0.1 | mM | 1 | mL |
| bFGF | 4 | ng/mL | 40 | μL |
| L-glutamine | 2 | mM | 1 | mL |
| 2-Mercaptoethanol** | 0.1 | mM | 182 | μL |

*Nonessential amino acid;
**added before use

TABLE 2

| Reagent | Final concentration | | Volume in 100 mL | |
|---|---|---|---|---|
| KnockOut ™ DMEM | 1X | | 83 | mL |
| KnockOut ™ SR | 15% | | 15 | mL |
| NEAA* | 0.1 | mM | 1 | mL |
| LIF | 10 | ng/mL | 100 | μL |
| L-glutamine | 2 | mM | 1 | mL |
| 2-Mercaptoethanol** | 0.1 | mM | 182 | μL |

*Nonessential amino acid;
**added before use

As shown in FIG. 3, as a result of culturing various cells on the polymer thin film, cell spheroid type cell aggregates for all the cells could be obtained within 24 hours of the culture without a limitation to particular cells.

### Example 4

### Immunofluorescent Staining of Cell Spheroids

The cell spheroids obtained from the cell culture substrate (dish) including the deposited polymer thin film manufactured in example 1 were stained through immunofluorescent staining. For the immunofluorescent staining, the cell spheroids were transferred on a cover glass coated with 0.1% gelatin, and then cultured for one day. The cells were fixed with 4% paraformaldehyde at room temperature for 10 minutes. After washing three times with dulbecco's phosphate buffered saline (DPBS), the cells were allowed to permeate 0.1% Triton X-100 at room temperature for 15 minutes, and then again washed three times with DPBS. In order to prevent a non-specific reaction of primary antibody, the cells were treated with 1% bovine serum albumin (BSA) for 10 minutes. The cells were allowed to react at 4° C. for 12 hours using primary antibody (CHEMICON) attached to tenascin diluted with DPBS/0.1% BSA. After washing, the cells were allowed to react at room temperature for 1 hour using secondary antibody (SantaCruz) with fluorescein isothiocyanate (FITC) attached thereto and dissolved in DPBS. After washing, the cells were counter-stained for 5 minutes with Hoechst 33342 (Invitrogen) diluted to 2 μg/ml in DPBS. The cover glass was fixed onto the slide glass using mounting medium, followed by analysis using the Nikon confocal microscope. The used antibodies are shown in Table 3.

TABLE 3

| | Catalog | | |
|---|---|---|---|
| Antibody | No. | Manufacturer | Dilution |
| anti-Tenascin | AB19011 | Chemicon | 1:100 |
| | | | (Immunostaining) |
| anti-rabbit IgG-FITC | SC-2012 | Santa Cruz | 1:200 |
| | | Biotechnology | (Immunostaining) |
| anti-β Catenin | SC-7963 | Santa Cruz | 1:200 |
| | | Biotechnology | (Western blot) |
| anti-survivin | 71G4B7 | Cell Signalling | 1:1000 |
| | | | (Western blot) |
| anti-GAPDH | SC-25778 | Santa Cruz | 1:200 |
| | | Biotechnology | (Western blot) |
| anti-rabbit IgG-HRP | NCI1460 | Thermo scientific | 1:5000 |
| | | | (Western blot) |
| anti-mouse IgG- | NCI1430 | Thermo scientific | 1:5000 |
| HRP | | | (Western blot) |

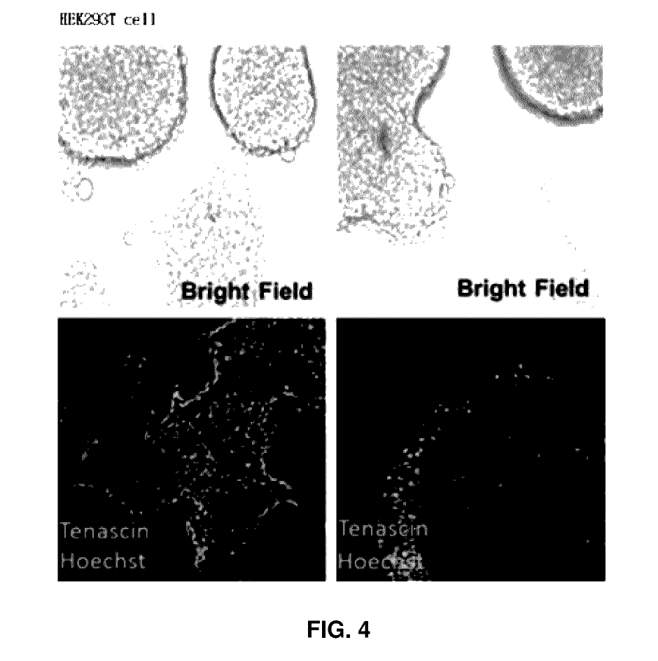

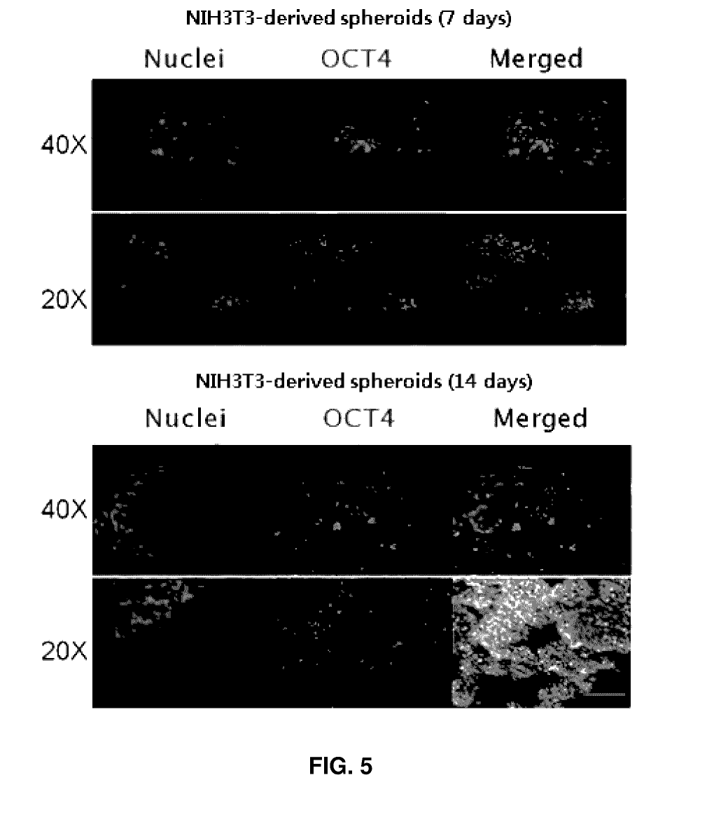

As a result, as shown in FIGS. 4 and 5, it was verified that the gene expression different as in the original cells was present inside the cell spheroids (FIGS. 4 and 5).

### Example 5

### Gene Analysis of Cell Spheroids

The cell spheroids obtained from the cell culture substrate (dish) including the deposited polymer thin film manufactured in example 1 were subjected to gene analysis. The total RNA (ribonucleic acid) was extracted from the formed cell spheroids using Hybrid-R™ kit (GeneAll). The cDNA synthesis was conducted using AccuPower RocketScript RT-PCR premix kit (Bioneer). The real-time polymerase chain reaction (RT-PCR) was conducted using Thermal Cyclers PCR machine (Bio-Rad) according to the following conditions. After cDNA synthesis at 42° C. for 60 minutes, a pre-denaturation at 95° C. for 5 minutes was conducted, and 35 cycles of denaturation at 95° C. for 30 seconds, annealing at 55° C. for 30 seconds, and extension at 72° C. for 10 seconds were finished, and then a final extension at 72° C. for 5 minutes was conducted. The used primers are shown in Table 4.

TABLE 4

| Gene | Forward primer | Reverse primer |
|---|---|---|
| human | GGGAAATGGGAGGGGTGCAA | TTGCGTGAGTGTGGATGGGA |
| Sox2 | AACAGG | TTGGTG |
| | (SEQ ID NO: 1) | (SEQ ID NO: 2) |
| human | CGTCTTCACCACCATGGAGA | CGGCCATCACGCCACAGTTT |
| Gapdh | (SEQ ID NO: 3) | (SEQ ID NO: 4) |
| mouse | TAGAGCTAGACTCCGGGC | TTGCCTTAAACAAGACCACG |
| Sox2 | GATGA | AAA |
| | (SEQ ID NO: 5) | (SEQ ID NO: 6) |
| mouse | TGTCCGTCGTGGATCTGAC | CCTGCTTCACCACCTTCTTG |
| Gapdh | (SEQ ID NO: 7) | (SEQ ID NO: 8) |

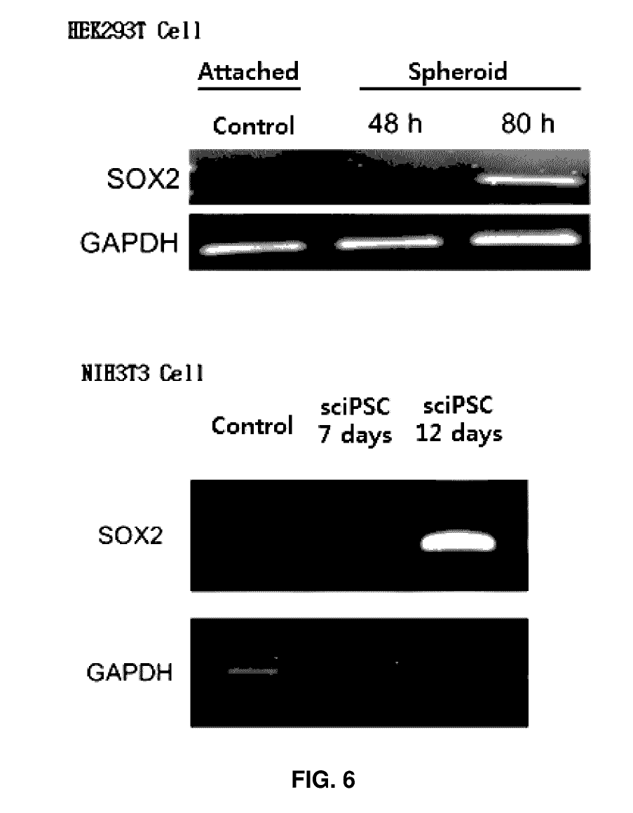

As a result, as shown in FIG. 6, the degrees of gene expression were varied compared with the cells cultured in the conventional PS cell culture dish (FIG. 6), and these results indicate that the cells were reprogrammed.

### Example 6

### Protein Analysis of Cell Spheroids

The cell spheroids obtained from the cell culture substrate (dish) including the deposited polymer thin film manufactured in example 1 were subjected to analysis at the protein level. The formed cell spheroids were lysed in RIPA buffer containing the protease inhibitor mixture (Thermo scientific), cultured on an ice for 15 minutes, and then centrifuged at 20,000 g for 10 minutes at 4° C. The supernatant was separated, and the protein concentration thereof was measured using Bradford assay kit (Bio-Rad). The separated proteins were fractionated on SDA PAGE, and the proteins were transferred to the polyvinylidene fluoride (PVDF) membrane (Millipore) at 4° C. using an electrophoresis apparatus. The membrane was allowed to react in PBS containing 0.1% Tween-20 and 5% skim milk for 30 minutes, and allowed to react with primary antibody (mouse anti-beta catenin, rabbit anti-survivin) diluted in PBS containing 5% skim milk at 4° C. for 12 hours. The membrane was washed three times with PBS containing 0.1% Tween-20, and then the film was allowed to react with secondary body (anti-mouse, anti-rabbit) with horseradish peroxidase (HRP) attached thereto at room temperature for 1 hour. The bands were visualized by the enhanced chemiluminescence (Thermo scientific) solution. The protein density of the bands was analyzed using ChemiDoc™ MP System (Bio-Rad), and was normalized to loading control (GAPDH) bands. The used antibodies are shown in Table 3.

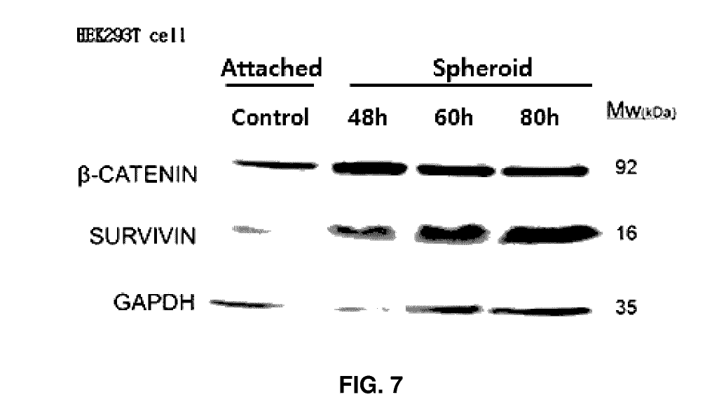

As a result, as shown in FIG. 7, the degrees of protein expression were varied compared with the cells cultured in the conventional PS cell culture dish (FIG. 7), and these results indicate that the cells were reprogrammed.

### Example 7

### Formation of Cell Spheroids of Mouse Embryonic Fibroblasts

From female mice (STOCK Tg(Nanog-GFP, Puro) 1Yam (No. RBRCO2290), RIKEN BRC) that were genetically modified to simultaneously express the green fluorescence protein (GFP) at the time of Nanog expression, the embryonic mice on day 16 after pregnancy were collected and somatic cells (mouse embryonic fibroblasts) were cultured. While the cells were cultured on the cell culture substrate (dish) including the deposited polymer thin film manufactured in example 1, for 7 days, the culture liquid was exchanged once. As the cell culture liquid, the mouse stem cell culture liquid containing culture ingredients shown in Table 2 was used. The cell spheroids formed on day 7 of the culture were fixed using a fixing liquid (4% paraformaldehyde) for the confocal microscopic observation, and the fixed cell spheroids were observed using the Zeiss confocal microscope.

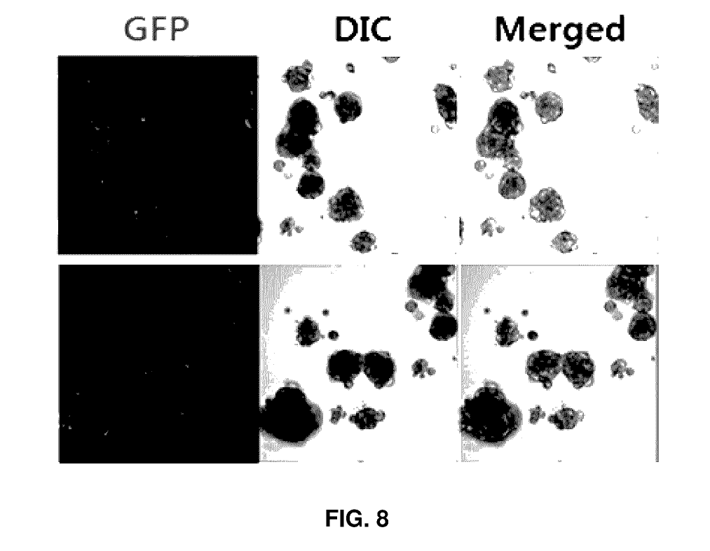

As a result, as shown in FIG. 8, it was verified that the green fluorescent proteins were expressed in the formed cell spheroids (FIG. 8). These results indicate that Nanog, which is one of the stem cell markers, was expressed in the cell spheroids.

### Example 8

### Cell Spheroid Forming Effect of Various Cyclosiloxane Polymers

Like in example 1, a cyclosiloxane (Sigma-Aldrich), as a monomer, on table 5 was put in a monomer container of a chemical vapor deposition reactor (iCVD, Daeki Hi-Tech Co., Ltd), and then heated at 70° C. The tert-butyl peroxide (TBPO, Sigma-Aldrich) as an initiator was put into an initiator container, and then maintained at room temperature. The deposition was carried out using a polystyrene cell culture plate as a substrate. The deposition was carried out for 40 minutes while the monomer and TBPO were allowed to flow into the chemical vapor deposition reactor at a rate of 1:1 (flow rate base, unit: sccm), the filament temperature in the reactor is 200° C., the substrate temperature in the reactor is 40° C., and the chamber pressure in the reactor is 200 mba, so that a cell culture substrate including a cyclosiloxane polymer deposited with about 200 nm thickness was manufactured.

TABLE 5
Cyclosiloxane monomer

| | |
|---|---|
| \textcircled{1} | 1,3,5-triisopropyl-1,3,5-trivinylcyclotrisiloxane |
| \textcircled{2} | 1,3,5,7-tetraisopropyl-1,3,5,7-tetravinylcyclotetrasiloxane |
| \textcircled{3} | 1,3,5,7,9-pentaisopropyl-1,3,5,7,9-pentavinylcyclopentasiloxane |
| \textcircled{4} | 1,3,5-tri-sec-butyl-1,3,5-trivinylcyclotrisiloxane |
| \textcircled{5} | 1,3,5,7-tetra-sec-butyl-1,3,5,7-tetravinylcyclotetrasiloxane |
| \textcircled{6} | 1,3,5,7,9-penta-sec-butyl-1,3,5,7,9-pentavinylcyclopentasiloxane |
| \textcircled{7} | 1,3,5-triethyl-1,3,5-trivinylcyclotrisiloxane |
| \textcircled{8} | 1,3,5,7-tetraethyl-1,3,5,7-tetravinylcyclotetrasiloxane |
| \textcircled{9} | 1,3,5,7,9-pentaethyl-1,3,5,7,9-pentavinylcyclopentasiloxane |
| \textcircled{10} | hexavinylcyclotrisiloxane |
| \textcircled{11} | octavinylcyclotetrasiloxane |
| \textcircled{12} | decavinylcyclopentasiloxane |

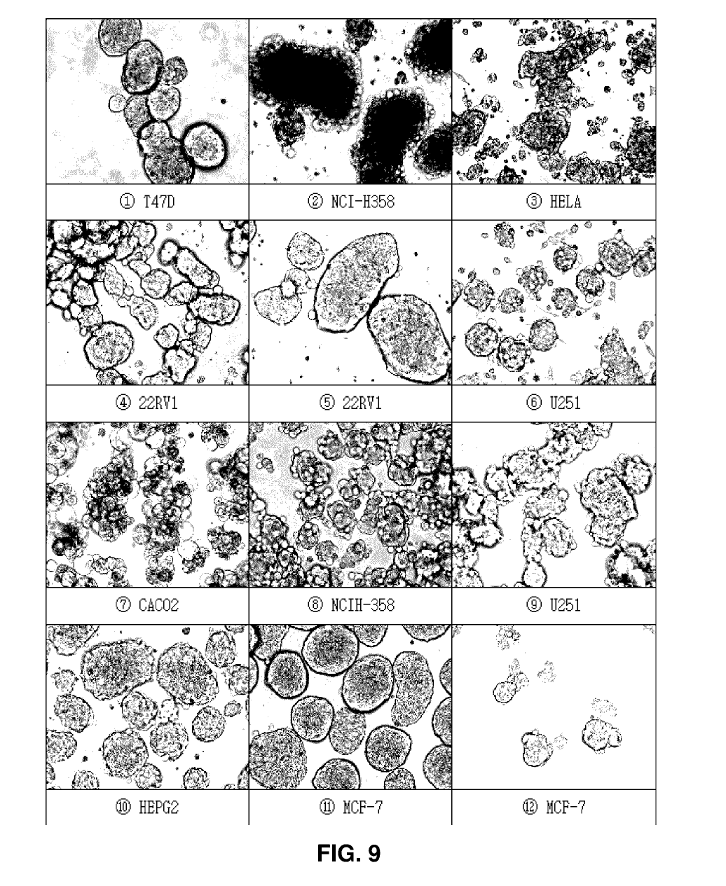

Thereafter, cells were cultured on the cell culture substrate including the deposited cyclosiloxane polymer thin film, and then it was checked whether or not cell spheroids were formed. The used cells were cancer cells as shown in FIG. 9. The cancer cells were cultured for 48 hours using a medium in which 10% (v/v) fetal bovine serum (Thermo) and 5% of 1% (w/v) penicillin/streptomycin (Gibco) were supplemented to the cell culture minimum medium.

The test results are shown FIG. 9. In FIG. 9, panel \textcircled{1} shows the results of culturing T47D cells on a polymer formed of 1,3,5-triisopropyl-1,3,5-trivinylcyclotrisiloxane; panel \textcircled{2} shows the results of culturing NCI-H358 cells on a polymer formed of 1,3,5,7-tetraisopropyl-1,3,5,7-tetravinylcyclotetrasiloxane; panel \textcircled{3} shows the results of culturing HELA cells on a polymer formed of 1,3,5,7,9-pentaisopropyl-1,3,5,7,9-pentavinyl cyclopentasiloxane; panel \textcircled{4} shows the results of culturing 22RV1 cells on a polymer formed of 1,3,5-tri-sec-butyl-1,3,5-trivinyl cyclotrisiloxane; panel \textcircled{5} shows the results of culturing 22RV1 cells on a polymer formed of 1,3,5,7-tetra-sec-butyl-1,3,5,7-tetravinyl cyclotetrasiloxane; panel \textcircled{6} shows the results of culturing U251 cells on a polymer formed of 1,3,5,7,9-penta-sec-butyl-1,3,5,7,9-pentavinyl cyclopentasiloxane; panel \textcircled{7} shows the results of culturing CACO2 cells on a polymer formed of 1,3,5-triethyl-1,3,5-trivinyl cyclotrisiloxane; panel \textcircled{8} shows the results of culturing NCI-H358 cells on a polymer formed of 1,3,5,7-tetraethyl-1,3,5,7-tetravinyl cyclotetrasiloxane; panel \textcircled{9} shows the results of U251 cells on a polymer formed of 1,3,5,7,9-pentaethyl-1,3,5,7,9-pentavinyl cyclopentasiloxane; panel \textcircled{10} shows the results of culturing HEPG2 cells on a polymer formed of hexavinyl cyclotrisiloxane; panel \textcircled{11} shows the results of culturing MCF-7 cells on a polymer formed of octavinyl cyclotetrasiloxane; and panel \textcircled{12} shows the results of culturing MCF-7 cells on a polymer formed of decavinyl cyclopentasiloxane.

As shown in FIG. 9, cell spheroids could be formed by culturing cells on the respective polymers formed of various cyclosiloxane compounds (FIG. 9).

### Example 9

### Cell Spheroid Forming Effect of Polymers

Into monomer containers of a chemical vapor deposition reaction (iCVD, Daeki Hi-Tech Co., Ltd), V4D4 (Sigma-Aldrich) as a first monomer and a second monomer selected from table 6 were put, respectively, and then heated. Here, the heating temperature was as follows. V4D4: 70° C.; monomer \textcircled{1}: 70° C.; monomer \textcircled{2}: 40° C.; monomer \textcircled{3}: 35° C.; monomer \textcircled{4}: not-heated (room temperature); monomer \textcircled{5}: 50° C.; monomer \textcircled{6}: 80° C.; monomer \textcircled{7}: 40° C.; monomer \textcircled{8}: 50° C.; monomer \textcircled{9}not-heated (room temperature); monomer \textcircled{10}: 40° C.; monomer \textcircled{11}: 50° C.; monomer \textcircled{12}: 50° C. The tert-butyl peroxide (TBPO, Sigma-Aldrich) as an initiator was put into an initiator container, and then maintained at room temperature. The deposition was carried out using a polystyrene cell culture plate as a substrate. At the time of depositing a copolymer of V4D4 and a second monomer, the deposition was carried out for 40 minutes while the V4D4 monomer, the second monomer, and TBPO were allowed to flow into the reactor at a rate of 9:1:9 (flow rate base, unit: sccm), the filament temperature in the reactor is 200° C., the substrate temperature in the reactor is 40° C., and the chamber pressure in the reactor is 250 mba, so that a cell culture substrate including a copolymer deposited with about 200 nm thickness was manufactured.

TABLE 6

| Second monomer for forming copolymer | | Purchased from |
|---|---|---|
| \textcircled{1} | 1,3,5-trivinyl-1,3,5-trimethylcyclotrisiloxane | Sigma-Aldrich |
| \textcircled{2} | Hexavinyldisiloxane | Gelest Inc. |
| \textcircled{3} | Glycicyl methacrylate | Sigma-Aldrich |
| \textcircled{4} | Perfluoro methacrylate | Sigma-Aldrich |
| \textcircled{5} | Benzyl methacrylate | Sigma-Aldrich |
| \textcircled{6} | 3,3,4,4,5,5,6,6,7,7,8,8,9,9,10,10,10- | Sigma-Aldrich |
| | heptadecafluorodecyl methacrylate | |
| \textcircled{7} | 4-vinylpyridin | Sigma-Aldrich |
| \textcircled{8} | Butyl methacrylate | Sigma-Aldrich |
| \textcircled{9} | Styrene | Sigma-Aldrich |
| \textcircled{10} | 1-vinylimidazole | Sigma-Aldrich |
| \textcircled{11} | Divinylbenzene | Sigma-Aldrich |
| \textcircled{12} | propargyl acrylate | Sigma-Aldrich |

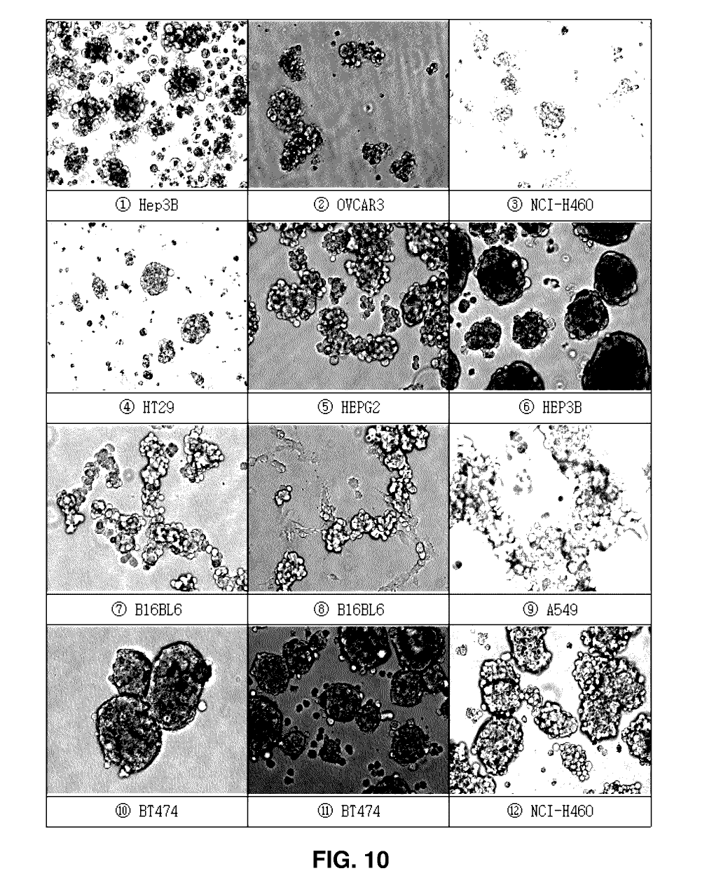

Thereafter, cells were cultured on the cell culture substrate including the deposited cyclosiloxane copolymer thin film, and then it was checked whether or not cell spheroids were formed. The used cells were cancer cells as shown in FIG. 10. The cancer cells were cultured for 48 hours using a medium in which 10% (v/v) fetal bovine serum (Thermo) and 5% of 1% (w/v) penicillin/streptomycin (Gibco) were supplemented to the cell culture minimum medium.

The test results are shown FIG. 10. In FIG. 10, panel \textcircled{1} shows the results of culturing Hep3B cells on a surface of a copolymer of 2,4,6,8-tetramethyl-2,4,6,8-tetravinyl cyclotetrasiloxane and 1,3,5-trivinyl-1,3,5-trimethyl cyclotrisiloxane; panel \textcircled{2} shows the results of culturing OVCAR3 cells on a surface of a copolymer of 2,4,6,8-tetramethyl-2,4,6,8-tetravinyl cyclotetrasiloxane and heaxavinyl disiloxane; panel \textcircled{3} shows the results of culturing NCI-H460 cells on a surface of a copolymer of 2,4,6,8-tetramethyl-2,4,6,8-tetra vinylcyclotetrasiloxane and glycidyl methacrylate; panel \textcircled{4} shows the results of culturing HT29 cells on a surface of a copolymer of 2,4,6,8-tetramethyl-2,4,6,8-tetravinyl-cyclotetrasiloxane and perfluoro methacrylate; panel \textcircled{5} shows the results of culturing HepG2 cells on a surface of a copolymer of 2,4,6,8-tetramethyl-2,4,6,8-tetravinylcyclotetrasiloxane and benzyl methacrylate; panel \textcircled{6} shows the results of culturing Hep3B cells on a surface of a copolymer of 2,4,6,8-tetramethyl-2,4,6,8-tetravinylcyclotetrasiloxane and 3,3,4,4,5, 5,6,6,7,7,8,8,9,9,10,10,10-heptafluorodecyl methacrylate; panel \textcircled{7} shows the results of culturing B16BL cells on a surface of a copolymer of 2,4,6,8-tetramethyl-2,4,6,8-tetravinylcyclotetrasiloxane and 4-vinylpyridine; panel \textcircled{8} shows the results of culturing B16BL6 cells on a surface of a copolymer of 2,4,6,8-tetramethyl-2,4,6,8-tetravinylcyclotetrasiloxane and butylmethacrylate; panel \textcircled{9} shows the results of culturing A549 cells on a surface of a copolymer of 2,4,6,8-tetramethyl-2,4,6,8-tetravinylcyclotetrasiloxane and styrene; panel \textcircled{10} shows the results of culturing BT474 cells on a surface of a copolymer of 2,4,6,8-tetramethyl-2,4,6,8-tetravinylcyclotetrasiloxane and 1-vinylimidazole; panel \textcircled{11} shows the results of culturing BT474 cells on a surface of a copolymer of 2,4,6,8-tetramethyl-2,4,6,8-tetravinylcyclotetrasiloxane and divinylbenzene; and panel \textcircled{12} shows the results of culturing NCI-H460 cells on a surface of a copolymer of 2,4,6,8-tetramethyl-2,4,6,8-tetravinylcyclotetrasiloxane and propargyl acrylate.

As shown in FIG. 10, cell spheroids could be formed by culturing cells on the respective copolymers formed of various cyclosiloxane compounds (FIG. 10).

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A cell culture substrate comprising a polymer formed of a cyclosiloxane compound.

2. The cell culture substrate of claim 1, wherein the cyclosiloxane compound is represented by chemical formula 1 below: 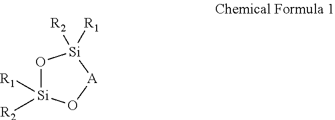
wherein
A is 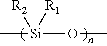
(n is an integer of 1-8);
R₁'s are each independently hydrogen or C₂₋₁₀ alkenyl, provided that at least two of R₁'s are C₂₋₁₀ alkenyl; and
R₂'s are each independently hydrogen, C₁₋₁₀ alkyl, C₂₋₁₀ alkenyl, halo, a metal element, C₅₋₁₄ heterocycle, C₃₋₁₀ cycloalkyl, or C₃₋₁₀ cycloalkenyl.

3. The cell culture substrate of claim 2, wherein the cyclosiloxane has (n+1) or (n+2) C₂₋₁₀ alkenyl at the R₁ positions.

4. The cell culture substrate of claim 1, wherein the cyclosiloxane compound is selected from the group consisting of
2,4,6,8-tetra(C₁₋₁₀)alkyl-2,4,6,8-tetra(C₂₋₁₀)alkenylcyclotetrasiloxane,
1,3,5-tri(C₁₋₁₀)alkyl-1,3,5-tri(C₂₋₁₀)alkenylcyclotrisiloxane,
1,3,5,7-tetra(C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
1,3,5-tri(C₁₋₁₀)alkyl-1,3,5-tri(C₂₋₁₀)alkenylcyclotrisiloxane,
1,3,5,7-tetra(C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
1,3,5-tri(C₁₋₁₀)alkyl-1,3,5-tri(C₂₋₁₀)alkenylcyclotrisiloxane,
1,3,5,7-tetra (C₁₋₁₀)alkyl-1,3,5,7-tetra(C₂₋₁₀)alkenylcyclotetrasiloxane,
1,3,5,7,9-penta(C₁₋₁₀)alkyl-1,3,5,7,9-penta(C₂₋₁₀)alkenylcyclopentasiloxane,
hexa(C₂₋₁₀)alkenylcyclotrisiloxane,
octa(C₂₋₁₀)alkenylcyclotetrasiloxane,
deca(C₂₋₁₀)alkenylcyclopentasiloxane, and
a combination thereof.

5. The cell culture substrate of claim 1, wherein the polymer formed of the cyclosiloxane compound is a copolymer formed of a first monomer, which is the cyclosiloxane compound, and a second monomer polymerizable with the first monomer.

6. The cell culture substrate of claim 5, wherein the second monomer is a cyclosiloxane compound different from the first monomer.

7. The cell culture substrate of claim 5, wherein the second monomer is a compound having a carbon double bond for polymerization with the first monomer.

8. The cell culture substrate of claim 7, wherein the second monomer is selected from the group consisting of siloxanes having a vinyl group, methacrylate-based monomers, acrylate-based monomers, aromatic vinyl-based monomers, acrylamide-based monomers, maleic anhydrides, silazanes or a cyclosilazanes having a vinyl group, C₃₋₁₀ cycloalkanes having a vinyl group, vinylpyrrolidones, 2-(methacryloyloxy)ethyl acetoacetate, 1-(3-aminopropyl)imidazole, vinylimidazoles, vinylpyridines, silanes having a vinyl group, and combinations thereof.

9. The cell culture substrate of claim 1, wherein the cell culture substrate is for manufacturing a cell spheroid type cell aggregate.

10. The cell culture substrate of claim 1, wherein the cell culture substrate is for manufacturing induced pluripotent stem cells.

11. A method for manufacturing a cell spheroid type cell aggregate, the method comprising a step for culturing cells on the cell culture substrate of claim 1.

12. The method of claim 11, wherein the cells are somatic cells, germ cells, cancer cells, or stem cells.

13. A method for manufacturing induced pluripotent stem cells, the method comprising a step for culturing differentiated cells on the cell culture substrate of claim 1.

14. The method of claim 13, wherein the differentiated cells are somatic cells or cancer cells.

15. (canceled)

16. The cell culture substrate of claim 1, wherein the cell culture substrate is for manufacturing cancer stem cells.


SEQUENCE LISTING (8 sequences)

SEQ ID NO: 1 - DNA, 26 bp, Artificial Sequence
  forward primer for human Sox2
gggaaatgggaggggtgcaaaacagg

SEQ ID NO: 2 - DNA, 26 bp, Artificial Sequence
  reverse primer for human Sox2
ttgcgtgagtgtggatgggattggtg

SEQ ID NO: 3 - DNA, 20 bp, Artificial Sequence
  forward primer for human Gapdh
cgtcttcaccaccatggaga

SEQ ID NO: 4 - DNA, 20 bp, Artificial Sequence
  reverse primer for human Gapdh
cggccatcacgccacagttt

SEQ ID NO: 5 - DNA, 23 bp, Artificial Sequence
  forward primer for mouse Sox2
tagagctagactccgggcgatga

SEQ ID NO: 6 - DNA, 23 bp, Artificial Sequence
  reverse primer for mouse Sox2
ttgccttaaacaagaccacgaaa

SEQ ID NO: 7 - DNA, 19 bp, Artificial Sequence
  forward primer for mouse Gapdh
tgtccgtcgtggatctgac

SEQ ID NO: 8 - DNA, 20 bp, Artificial Sequence
  reverse primer for mouse Gapdh
cctgcttcaccaccttcttg